(19) United States
(12) Patent Application Publication
(10) Pub. No.: US 2025/0041535 A1
(43) Pub. Date: Feb. 6, 2025

(54) Drug Delivery Arrangement

(71) Applicant: Sanofi, PARIS (FR)

(72) Inventors: Max Davis, Melbourn, Hertfordshire (GB); Thomas Mark Kemp, Melbourn, Hertfordshire (GB); Tom Lever, Melbourn, Hertfordshire (GB); Max Wehebrink, Melbourn, Hertfordshire (GB); Robbie Wilson, Melbourn, Hertfordshire (GB)

(21) Appl. No.: 18/718,731

(22) PCT Filed: Dec. 13, 2022
(86) PCT No.: PCT/EP2022/085635

§ 371 Date: Jun. 11, 2024

(30) Foreign Application Priority Data
Dec. 15, 2021 (EP) 21315273.9

(51) Int. Cl. A61M 5/46; A61M 5/20; A61M 5/34

(52) U.S. Cl. CPC A61M 5/46; A61M 5/20; A61M 5/347

(57) ABSTRACT

A drug delivery arrangement includes a drug delivery device with a housing adapted to receive a needle that is configured to pierce a skin of a user. The drug delivery device comprises a drive energy source, and is configured to perform a drug delivery operation using the energy obtainable from the drive energy source. The drug delivery device is an autoinjector. An injection depth adjusting member is connectable to or connected to a device member of the drug delivery device. The injection depth adjusting member is configured to provide an adjuster bearing surface with which the drug delivery arrangement bears on the skin during use when the injection depth adjusting member is connected to the device member. When connected to the device member, the injection depth adjusting member is configured to define the injection depth by which the needle can penetrate the skin.

### CROSS REFERENCE TO RELATED APPLICATIONS

The present application is the national stage entry of International Patent Application No. PCT/EP2022/085635, filed on Dec. 13, 2022, and claims priority to Application No. EP 21315273.9, filed on Dec. 15, 2021, the disclosures of which are incorporated herein by reference.

### BACKGROUND

The present disclosure relates to a drug delivery arrangement, in particular a drug delivery arrangement suitable to provide an adjustable injection depth.

In drug delivery devices, drug is often delivered to a user via a needle which pierces the skin of the user (or patient). The needle pierces into the skin with a certain injection depth. Usually, the injection depth of a particular device is set by the design of the device. For some users, the injection depth, for which a particular device is designed, is not suitable. Children, for example, usually have less tissue available below the skin to be pierced as compared to grown-ups. Hence a reduced injection depth would be advantageous for children.

### SUMMARY

It is an object of the present disclosure to facilitate improvements associated with drug delivery devices, particularly with respect to injection depth adjustments.

This object is achieved by subject-matter disclosed, for example by the subject-matter defined in the appended independent claim. Advantageous refinements and developments are subject to dependent claims and/or set forth in the description below.

One aspect of the present disclosure relates to a drug delivery arrangement. The drug delivery arrangement comprises a drug delivery device. The drug delivery device may be provided to dispense drug or medicament from a medicament container. The drug delivery device comprises a housing. The housing is adapted to receive a needle and, preferably, the medicament container. The needle and/or the medicament container may be arranged in the housing. The needle is expediently configured to pierce a skin of a user. Through the needle, the medicament may be administered to the user, e.g. into the user's tissue. The drug delivery device may comprise a drive energy source, e.g. a drive spring or another type of energy source such as a gas reservoir. The drug delivery device is configured to perform a drug delivery operation, e.g. using energy obtainable from the drive energy source. The drive energy source may be configured to provide energy for the drug delivery operation of the drug delivery device. The energy may be used to drive a drive member, e.g. a plunger rod, of the drug delivery device in order to dispense drug from the medicament container. For the drug delivery operation, the drive member may be displaced in a distal direction relative to the housing by the energy provided by the drive energy source.

The drug delivery arrangement further comprises an injection depth adjusting member. The injection depth adjusting member is connectable to or connected to a device member of the drug delivery device. The injection depth adjusting member may be configured to adjust or define the injection depth of the drug delivery device or the drug delivery arrangement. The injection depth may be the distance by which the needle protrudes into the skin, such as during the drug delivery operation. The injection depth adjusting member is configured to provide an adjuster bearing surface with which the drug delivery arrangement bears on the skin during use, e.g. during the drug delivery operation and/or when the injection depth adjusting member is connected to the device member. A “bearing surface” of the device or the arrangement may be adjacent to a location where the needle pierces the skin for the drug delivery operation when the drug delivery arrangement or the device is used for the drug delivery operation. The injection depth adjusting member, e.g. when connected to the device member, may be configured to adjust or define or may adjust or define the injection depth by which the needle can penetrate the skin, e.g. for or during the drug delivery operation. For example, the injection depth adjusting member may reduce the injection depth by which the needle can penetrate into the skin for the drug delivery operation, e.g. by the distance between the adjuster bearing surface and a bearing surface which the drug delivery device may have for a drug delivery operation without the injection depth adjusting member. The drug delivery device of the arrangement may be a fully functional drug delivery device which can be used with the injection depth adjusting member and without the injection depth adjusting member. Preferably, the injection depth adjusting member merely adjusts the injection depth, e.g. to a desired injection depth. When the injection depth adjusting member is connected to the device member, it may be axially secured relative to the device member, preferably against axial movement in both directions (e.g. proximal and distal) or at least against axial movement which would increase the injection depth, e.g. movement in the proximal direction. That is to say, the injection depth adjusting member may be fixed relative to the device member such that the distance between a bearing surface of the drug delivery device and the adjuster bearing surface is constant or at least not reduced during use of the drug delivery arrangement, which may comprise performing a drug delivery operation with the injection depth adjusting member being connected to the device member.

In one embodiment, the drug delivery device is an autoinjector. In an autoinjector the energy for the drug delivery operation may be prestored in the drive energy source. That is to say, the user does not have to provide the energy for the drug delivery operation, e.g. when preparing the device for use. Rather, this energy may be preloaded into the system by the manufacturer. For example, a drive spring may be pre-stressed or pre-biased to provide the energy for the drug delivery operation.

In one embodiment, the device member is arranged to provide a member bearing surface with which the drug delivery device can bear on or is intended to bear on the skin, e.g. the user's skin and/or when the drug delivery device is operated to perform the drug delivery operation, preferably without the injection depth adjusting member being connected to the drug delivery device. That is to say, the member, which provides the bearing surface of the device in case the drug delivery device is operated without the injection depth adjusting member connected to the drug delivery device, may be the same member to which the injection depth adjusting member is connectable or connected, e.g. to form a drug delivery device with adjusted injection depth. The device member may be a static member of the drug delivery device, e.g. a member which does not move relative to a housing of the device for the drug delivery operation, or a movable member of the drug delivery device.

In one embodiment, the (respective) bearing surface, e.g. adjuster bearing surface, device bearing surface and/or member bearing surface, is oriented in a distal direction, in case of the injection depth adjusting member expediently when the injection depth adjusting member is connected to the device member. The injection depth adjusting member, when connected to the device member, may form the distal end of the drug delivery arrangement (which then may be a drug delivery device with adjusted or modified injection depth), e.g. before the drug delivery operation is commenced and/or before the needle pierces the skin. A cap potentially covering a distal end of the device or the arrangement may then have been removed already.

In one embodiment, the injection depth adjusting member is releasably connected to or releasably connectable to the device member. In other words, the injection depth adjusting member may be preinstalled to the device member and may be removable from the device member. The injection depth adjusting member can be removed from the drug delivery device to vary, e.g. to increase, the injection depth. The drug delivery arrangement may be configured such that, when the injection depth adjusting member has been removed or disconnected from the drug delivery device, it cannot be reconnected, e.g. by providing a connection feature which breaks when the injection depth adjusting member is removed from the drug delivery device. This has the advantage that the user knows that he cannot attempt to reconnect the adjusting member. When the injection depth adjusting member has been removed or disconnected from the drug delivery device, the device may be ready to initiate a drug delivery operation. Preferably, one or more safety mechanisms which were available to protect the user before the disconnection may no longer be present. Hence, preventing the user from an attempt to reattach the injection depth adjusting member, which might be dangerous, may increase user safety. Alternatively, the injection depth adjusting member may be disconnected from and reconnected to the device member.

In one embodiment, the injection depth adjusting member is disconnected from the drug delivery device, e.g. when the drug delivery arrangement is provided by the manufacturer. Preferably, the user can decide whether to connect the injection depth adjusting member to the device member in order to adjust the injection depth to a desired injection depth or not. Once the injection depth adjusting member has been connected to the device member, the connection may be releasable or irreleasable (non-releasable). If the connection is non-releasable, the user knows that it does not make sense to tamper with the injection depth adjusting member after having connected it to the device member. This is because the adjusting member could not be reused if it were attempted to disconnected from the device member, because, on account of the non-releasable connection, the one of the parts would be permanently damaged, if the attempt were successful.

In one embodiment, the drug delivery arrangement comprises a plurality of injection depth adjusting members. The injection depth adjusting members may be configured to provide different injection depths.

The injection depth adjusting members may be provided disconnected from the device member and/or be connected to the device member, e.g. to achieve the desired injection depth. For example, the user can decide which one of the plurality of adjusting members he wants to use based on the desired injection depth and connect that injection depth adjusting member from the plurality of injection depth adjusting members to the device member which achieves the desired injection depth when connected to the device member. For example, a first one of the adjusting members can achieve a first injection depth when connected to the device member and the second one of the adjusting members can achieve a second injection depth when connected to the device member where the second and first injection depths are different.

The plurality of injection depth adjusting members may be provided connected to the device member or preinstalled to the device member. For example, the injection depth adjusting members may be part of an injection depth adjusting member assembly. The injection depth adjusting members may be interconnected with one another. The injection depth adjusting members may be releasably interconnected with each other and/or to the device member such that one or a plurality of injection depth adjusting members can be disconnected or released from the assembly or the device member, e.g. to achieve the desired injection depth. Each of the injection depth adjusting members may be configured to provide its own adjuster bearing surface, e.g. when the surface is exposed. Different adjuster bearing surfaces, when exposed, may achieve different injection depths. When disconnecting one or more injection depth adjusting members from the device member or from the remainder of the injection depth adjusting member assembly, the injection depth may be increased (as the member is removed). Injection depth adjusting members of the assembly may be arranged in a stacked fashion. Only the most proximal one of the injection depth adjusting members of the stack may be connected directly to the device member. The other injection depth adjusting members may be connected directly to that adjusting member of the stack which proximally adjoins the respective adjusting member.

In one embodiment, the injection depth adjusting member is connected to or connectable to a distal portion of the drug delivery device. A portion of the device member which may form a connection interface with a connection feature of the injection depth adjusting member may be positioned close to or at a distal end of the device. The portion of the device member may protrude distally from the housing or be a distal portion of the housing.

In one embodiment, the device member is a member different from the housing. Alternatively, the device member may be the housing. If the device member is a member different from the housing, the device member may be movable relative to the housing. For example, the device member may protrude distally from the housing. The device member may be movable relative to the housing from an initial or first position in a proximal direction into a second position, e.g. in an operation position. In the second position, the drug delivery operation may be triggerable or may already have commenced. The device member may be movable relative to the housing with and/or without the injection depth adjusting member being connected to the device member, e.g. from the first into the second position. The device member may have to be moved relative to the housing to trigger or to enable triggering of the drug delivery operation. That is to say, movement of the device member relative to the housing may be necessary such that a drug delivery operation can be conducted by the drug delivery device. The device member may be the trigger member which, when moved, directly triggers initiation of the drug delivery operation. Alternatively, a separate trigger member may be provided and the movement of the device member may only enable the trigger member to be operated, e.g. by removing a mechanical block which prevents movement of the trigger member unless the device member is moved.

In one embodiment, the drug delivery device comprises a needle shroud. The device member may be the needle shroud. The needle shroud may be provided to cover the needle. The needle shroud may be provided to cover the needle before the needle pierces the skin and/or after the needle has been removed from the skin, e.g. after completion of the drug delivery operation. Before the drug delivery operation is commenced, the needle shroud may protrude distally from the housing, e.g. to cover the tip of the needle (such as by axially extending beyond the tip of needle in the distal direction, e.g. at least with a bearing surface of the needle shroud). For the drug delivery operation, the needle shroud may be displaced proximally relative to the housing. After completion of the drug delivery operation, the needle shroud may be moved distally relative to the housing, e.g. to cover the tip of needle and/or into a third position relative to the housing. The drug delivery device may comprise a shroud spring. The shroud spring may be operatively coupleable to or coupled to the needle shroud in order to move the needle shroud, e.g. into the distal direction relative to the housing. The force of the shroud spring may have to be overcome in order to move the needle shroud in the proximal direction away from the first position. In a final position or third position, e.g. after the drug delivery operation has been completed and the device or the arrangement has been removed from the skin, the needle shroud may be locked against proximal movement with respect to the housing, such as by a locking mechanism.

In one embodiment, the device member is an activation member. The activation member is a member which may have to be moved relative to the housing in order to enable triggering of the drug delivery operation or to trigger the drug delivery operation. Enable triggering of the drug delivery operation may comprise that in addition to movement of the activation member another member such as a trigger member has to be actuated, e.g. a trigger button has to be pressed. Alternatively, the device member itself may be the trigger member. Thus, when the device member reaches the second position, the drug delivery operation may be initiated, e.g. resulting in the plunger rod being moved in the distal direction relative to the housing.

In one embodiment, the needle, e.g. when received in the housing, is axially fixed relative to the housing. That is to say, axial movement of the needle relative to the housing may be prevented, preferably in the distal direction and/or in the proximal direction. The needle may be received in the housing of the drug delivery device. Having a needle which is fixed relative to the housing facilitates adjusting the injection depth to a desired injection depth because positional changes of the needle relative to the housing cannot influence the injection depth. The presently disclosed concepts do also apply to devices comprising or provided to retain a movable needle which is configured to move relative to the housing and/or the device member, e.g. driven by a spring, for piercing the skin and/or for the drug delivery operation.

In one embodiment, the drug delivery device comprises a medicament container holding a medicament. The needle may be in or may be brought into fluid communication with an interior of the medicament container. The needle may be integrated into the medicament container. The medicament, e.g. a liquid medicament, is expediently arranged in the interior of the container. The medicament container may be a syringe, e.g. a syringe with a preinstalled needle, such as a staked needle. Alternatively, the medicament container may be a cartridge, which may have to be brought into fluid communication with a separate needle unit, e.g. by piercing a cartridge septum with the needle of the needle unit.

In one embodiment, the drug delivery device comprises a safety member. The safety member may be a removable safety member. That is to say, it may be removed from the drug delivery device. The safety member may be operatively connected to the device member or the needle shroud. The safety member may be accessible for the user to be removed by the user before the drug delivery operation. The safety member may be provided or arranged to prevent a movement of the device member or the needle shroud relative to the housing, e.g. into a position which is required for the drug delivery operation to be initiated, preferably unless the safety member is removed. The movement of the device member or needle shroud relative to the housing, e.g. in the proximal direction, may be prevented unless the safety member is removed. For example, the safety member may be arranged to abut the housing before the device member or the needle shroud reaches the second position, which may be required for initiation of the drug delivery operation, and/or before the needle is able to pierce the skin. When the safety member has been removed, movement of the device member may no longer be prevented by the safety member. In this case, the drug delivery operation may be initiated when the device member or the needle shroud is in the or reaches the second position and/or when the needle pierces the skin. Having a safety member in place is particularly advantageous, if the injection depth adjusting member is connected by the user to the device member or otherwise manipulated (e.g. directly) by the user to achieve the desired injection depth. Hence, after connection of the injection depth adjusting member to the device member (or if the user decides that no adjusting member is required) or after a manipulation of the injection depth adjusting member has been completed, the safety member may be removed: Preferably, the drug delivery operation can be only commenced after the removal. The safety member may be different from a cap of the drug delivery device. That is to say, it may still be present after the cap has been removed. The cap and/or the safety member may have to be removed from the drug delivery device to expose a bearing surface (e.g. the member bearing surface or the adjuster bearing surface).

In one embodiment, the injection depth adjusting member, when connected to the device member, is rigidly connected to the device member. That is to say, a position of the injection depth adjusting member (i.e. rotational position and/or axial position) relative to the device member is expediently constant and/or cannot be changed when the member is connected to the device member.

In one embodiment, the injection depth adjusting member, when connected to the device member, is movable relative to the device member. Hence, when the device member and the injection depth adjusting member are connected, the injection depth can still be adjusted by moving the injection depth adjusting member relative to the device member. The relative position between the adjusting member and the device member may be stable enough (e.g. despite the relative movability) so as to withstand a force required to move the device member or needle shroud proximally from the first position towards and/or into the second position, e.g. an actuation force. That is to say, the connection between the injection depth adjusting member and the device member may be stable enough so as to react the force of the shroud spring (which may increase during the movement of the needle shroud) and/or an actuation force which has to act on the injection depth adjusting member in order to enable initiation the drug delivery operation or initiate the drug delivery operation, for example. In this way, one bearing surface may be used for different injection depths (e.g. by continuously varying the injection depth by the movement). On the contrary, if the injection depth adjusting member is rigidly connected to the device member, different bearing surfaces (e.g. member bearing surface and adjuster bearing surface) expediently correspond to different (e.g. discrete) injection depths.

In one embodiment, the injection depth adjusting member, when connected to the device member, is threadedly connected to the device member. The injection depth adjusting member may be rotatable relative to the device member when connected to the device member. Via the threaded connection, the injection depth adjusting member may be axially displaced when rotated relative to the device member to adjust the injection depth while the injection depth adjusting member is connected to the device member. The threaded connection between the device member and the injection depth adjusting member may be formed by a threaded interface between the device member and the injection depth adjusting member. The threaded connection may be formed by means of at least one self-locking thread. In other words, the threaded connection may be self-locking. A self-locking threaded connection is a connection which, if only an axial load is applied to one of the members which are threadedly connected by the self-locking threaded connection, this axial load does not result in relative rotational movement between the members threadedly connected via the self-locking threaded connection. Hence, even though subjected to an axial load, the relative axial position between the members coupled by the self-locking threaded connection is maintained under the axial load. The pitch of the thread for the threaded connection may be chosen appropriately small in order to achieve a self-locking threaded connection.

In one embodiment, the drug delivery arrangement comprises a selection mechanism. The selection mechanism may be operable, e.g. by the user, to select a desired injection depth, e.g. from a plurality of possible injection depths. For example, one injection depth may be achieved or defined by the member bearing surface and one may be achieved by the adjuster bearing surface, which are expediently axially offset from each other. The selection mechanism may comprise a selection member. The selection member may be movable, e.g. by the user. The selection member may be movable relative to the housing and/or relative to the device member. The selection member may be movable in order to select the desired injection depth. The selection member may be rotatable relative to the housing to select the desired injection depth. The selection member may be movable, e.g. rotatable, from a first position to a second position, e.g. relative to the housing and/or the device member. The first position may be a position for a first injection depth and the second position may be a position for a second injection depth, where the second injection depth preferably is different from the first injection depth. The user may choose from a plurality of discrete injection depths, e.g. by disconnecting one or more preinstalled or connected injection depth adjusting members or leaving all preinstalled or connected injection depth adjusting members in place on the device member. Alternatively, the user may vary the injection depth continuously, e.g. by moving the injection depth adjusting member relative to the device member via the selection member.

In one embodiment, the selection member is operatively coupled to the injection depth adjusting member such that movement of the selection member is converted into movement of the injection depth adjusting member, e.g. relative to the device member and/or the housing.

In one embodiment, the selection member is rotationally locked to the injection depth adjusting member. Thus, rotation of the selection member results in rotation of the injection depth adjusting member.

In one embodiment, the selection member is movable, e.g. rotatable, relative to the injection depth adjusting member and/or the device member, e.g. when it is moved from the first position to the second position.

In one embodiment, the selection member is removably connected to the drug delivery device device.

In one embodiment, the selection member is removable or disconnectable from the drug delivery device. In the first position, the selection member may be arranged such that the injection depth adjusting member is removed or disconnected from the device member together with the selection member. For example, during removal or detachment of the selection member, the selection member may be operatively coupled to the injection depth adjusting member, e.g. axially locked to the adjusting member. When the injection depth adjusting member has been removed, a bearing surface, e.g. the adjuster bearing surface of another injection depth adjusting member or the member bearing surface of the device member, may be exposed. In the second position, the selection member may be removed without the injection depth adjusting member such that the injection depth adjusting member remains connected to the device member. Thus, the adjuster bearing surface may form the bearing surface for the drug delivery operation. In the second position, the selection member may be operatively decoupled from the injection depth adjusting member. In the second position of the selection member, a selection feature, e.g. a inwardly directed protrusion, may angularly overlap with a surface of the injection depth adjusting member facing in the proximal direction. In this way, the selection member, when removed in the distal direction, will cooperate with the surface via the selection feature, thereby simultaneously removing the injection depth adjusting member. In the second position, the selection feature may axially move beyond the injection depth adjusting member, e.g. because of a slot in the injection depth adjusting member which permits the selection feature to move axially along the injection depth adjusting member without interaction or substantial interaction. Thus, in the second position, the injection depth adjusting member may stay in place on the device member.

In one embodiment, the drug delivery arrangement comprises one or more visual and/or tactile indications for the position of the selection member, e.g. for the first position and the second position. Alternatively or additionally, the drug delivery arrangement may comprise one or more visual and/or tactile indications on the currently selected injection depth, e.g. depending on the position of the selection member and/or the injection depth adjusting member relative to the housing and/or depending on the position of the selection member relative to the injection depth adjusting member. The indication, e.g. a visual indication, may be provided by the selection member, by the injection depth adjusting member, by the device member, and/or by the housing or by a combination of any one of these components.

In one embodiment, the selection member is secured with respect to axial movement relative to the housing, e.g. when being moved from the first position to the second position. Hence, the selection member may be axially locked relative to the housing, e.g. during the selection procedure for selecting the desired injection depth. The connection to the housing may be releasable such that the selection member may be removed from the drug delivery device. In one embodiment, the drug delivery device or the drug delivery arrangement comprises a cap. The cap may cover a distal end, e.g. the needle end, of the drug delivery device or the arrangement and be removable or detachable from the drug delivery device or the arrangement.

The cap may cover and/or receive the injection depth adjusting member, which is connected to the device member. When the cap is removed, a needle passage opening of the drug delivery device or the arrangement may become exposed. The needle passage opening may be the opening through which the needle may protrude to pierce the skin may be covered by the cap before it is removed. The opening may be an opening in the device member or the injection depth adjusting member.

In one embodiment, the selection member is integrated into the cap or the cap is the selection member. The selection member may be removed together with the cap from the drug delivery arrangement.

In one embodiment, the selection member is different from the cap of the drug delivery device or the arrangement. Particularly, when the cap is removed, the selection member may remain connected to the drug delivery device. The selection member may become available for user manipulation for selecting the injection depth after removal of the cap, e.g. only after removal. The selection member may be manipulatable by the user to select the desired injection depth, e.g. by moving the selection member appropriately. After completion of the selection procedure, the selection member may be removed from the drug delivery device. Having a separate selection member facilitates to use a standard or not modified cap for the device and/or standard or proven interfaces of the cap to the device.

In one embodiment, the selection member is arranged to prevent access to and/or to prevent user manipulation of the injection depth adjusting member when the selection member is connected to the drug delivery device. The selection member may have a surface defining a distal end of the drug delivery arrangement and/or a surface being arranged to radially (preferably also distally) cover a bearing surface, e.g. the adjuster bearing surface.

In one embodiment, the drug delivery arrangement is configured to provide a plurality of different injection depths.

In one embodiment, the cap comprises a needle shield remover. The needle shield remover is, preferably, axially locked to a needle shield, which may cover the needle, e.g. when the cap is in place on the drug delivery device or the drug delivery arrangement. Hence, the needle shield may be removed together with the cap from the drug delivery device. The needle shield may cover the distal end of the needle (the needle tip) when connected to the needle.

In one embodiment, the adjuster bearing surface and/or the member bearing surface is positioned outside of the housing, e.g. at all times and/or in all positions of the device member and/or the injection depth adjusting member (when it is connected to the drug delivery device) relative to the housing.

In one embodiment, the injection depth adjusting member defines a needle passage opening. The needle tip may pass through the needle passage opening for piercing the skin. The diameter of needle passage opening of the (respective) injection depth adjusting member may be adjusted to, e.g. be equal to, the diameter of a needle passage opening of the device member or needle shroud. The diameter of the needle passage opening of the injection depth adjusting member may be greater than or equal to the one of the needle passage opening of the device member or needle shroud, for example.

In one embodiment, the injection depth adjusting member is connected to the device member such that it acts as a distal spacer or distal shim.

In one embodiment, the injection depth adjusting member is directly connected to the device member.

In one embodiment, the injection depth adjusting member has a ring-like portion. The thickness (or axial extension) of the ring-like portion may define the distance by which the injection depth is adjusted or changed by the injection depth adjusting member.

In one embodiment, the injection depth adjusting member is connected to a distal portion of the device member, e.g. a portion of the device member protruding from the housing in the distal direction, e.g. in the initial or first position of the device member relative to the housing.

In one embodiment, the injection depth adjusting member is connected to the device member in the region of a cylindrical portion of the device member. The device member may, in addition to the cylindrical portion have one or more arms which extend proximally from the cylindrical portion. The cylindrical portion may be the distal portion of the device member.

Features described above and below in conjunction with different embodiments or aspects can be combined with one another, even if such a combination is not explicitly disclosed herein above or below. Further features, advantages and expediencies of the disclosure and, particularly, of the proposed concepts will become apparent from the following description of the exemplary embodiments in conjunction with the drawings.

### BRIEF DESCRIPTION OF THE FIGURES

FIGS. 1A to 1D illustrate an embodiment of a drug delivery device.

FIG. 2 illustrates an embodiment of a drug delivery arrangement by way of different representations.

FIGS. 3A through 3D illustrate another embodiment of a drug delivery arrangement.

FIG. 4 illustrates another embodiment of a drug delivery arrangement.

FIG. 5 illustrates another embodiment of a drug delivery arrangement.

FIGS. 6A and 6B illustrate another embodiment of a drug delivery arrangement.

FIGS. 7A to 7D illustrate another embodiment of a drug delivery arrangement.

FIGS. 8A to 8E illustrate further embodiments of a drug delivery arrangement.

FIGS. 9A to 9E illustrate further embodiments of a drug delivery arrangement.

### DETAILED DESCRIPTION

Identical elements, elements of the same kind and identically or similarly acting elements may be provided with the same reference numerals in the drawings.

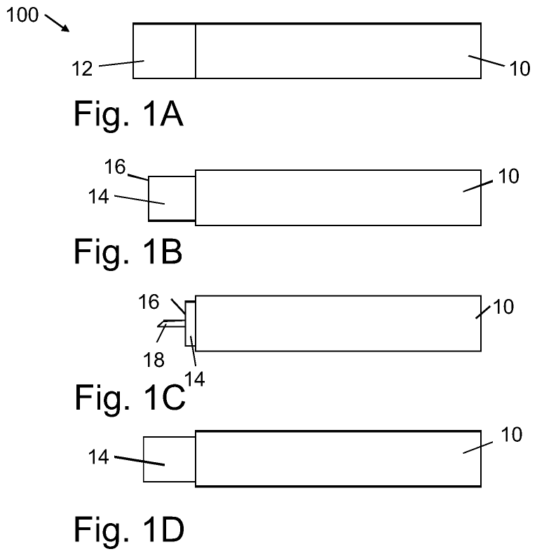

FIGS. 1A through 1D illustrate an embodiment of a drug delivery device 100. The device 100 is suitable as the device in the drug delivery arrangements described further above and below. The figures show the device 100 in different stages during its operation.

FIG. 1A shows the drug delivery device 100 in an initial or as delivered state. The drug delivery device comprises a housing 10. The housing is provided to retain and/or retains a medicament container (not explicitly shown in these figures) in its interior. Medicament, e.g. liquid medicament, is arranged in the medicament container. The housing is provided to retain and/or retains a needle. In other words, a needle may be arranged or arrangeable in the housing. The needle can be an integral part of the medicament container, e.g. (permanently or releasably) connected to a medicament container body, or separate from the medicament container. In the first case, the medicament container may be a syringe. In the second case, the medicament container may be a cartridge. In case a cartridge is used as medicament container, initially, the medicament container and the needle can be fluidly disconnected and fluid communication between the medicament container interior and the needle is only established during operation of the drug delivery device. A drive mechanism provided to drive a drug delivery operation is expediently provided in the housing 10 (not explicitly shown). The drive mechanism comprises a plunger rod (not explicitly shown). The drug delivery device further comprises a drive energy source, e.g. a drive spring, such as a compression spring, (not explicitly shown). The drive energy source is arranged to drive the plunger rod in a distal direction relative to the medicament container during the drug delivery operation. During this movement, a stopper, which is movably retained in the medicament container and may seal the medicament container proximally, can be displaced towards an outlet of the medicament container to dispense the drug or medicament retained within the medicament container through the outlet. The outlet may be formed or defined by the needle. Other potential drive energy sources different from a spring comprise an electrical power cell or battery for driving the plunger rod by a motor or a reservoir suitable to provide gas pressure, where the gas pressure can be used to drive the drug delivery operation. The drug delivery device is an autoinjector. The energy for driving the drug delivery operation in an autoinjector may be provided by components integral to the drug delivery device and does not have to be loaded into the device by the user during the operation of the device as is the case in many spring driven pen-type variable dose injectors, where, usually, the energy is loaded into the spring by the user during a dose setting procedure. The drug delivery device expediently is a single shot device, i.e. it is provided to dispense only one dose. The drug delivery device may be disposable drug delivery device, that is to say a device which is disposed of after its use. The device may be a pen-type device. The medicament container and/or the needle can be axially secured within the drug delivery device, e.g. within the housing, or can be movable relative to the housing, e.g. for piercing the skin. In the first case, the user may have to perform the movement for piercing the skin with the needle. In the second case, piercing of the needle may be driven by a needle insertion mechanism of the drug delivery device.

“Distal” is used herein to specify directions, ends or surfaces which are arranged or are to be arranged to face or point towards a dispensing end of the drug delivery device or the device arrangement or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or the delivery arrangement or components thereof. The distal end may be the end closest to the dispensing end and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle is arranged or a needle or needle unit is or is to be mounted to the device, for example.

As depicted in FIG. 1A, the drug delivery device further comprises a cap 12. The cap is arranged at the distal end of the drug delivery device 100. The cap is detachably connected to the remainder of the device, e.g. to the housing and/or another component or member of the drug delivery device. The cap, e.g. with a distally oriented surface, covers a distal end of the remainder of the drug delivery device and/or a needle passage opening through which the needle, e.g. the distal needle tip, may pass to pierce the skin from the interior of drug delivery device during or for the drug delivery operation. The cap can comprise a needle shield remover, which engages a needle shield which covers the needle such that the needle shield remover is removed from the needle together with the cap, e.g. when the cap is detached or disconnected from the device (not explicitly shown). The housing 10 expediently covers the majority of the length of the drug delivery device, e.g. 60% or more or 70% or more or 80% or more of the entire length of the drug delivery device 100 (with the cap 12 attached and/or with the cap detached).

FIG. 1B shows the drug delivery device of FIG. 1A with the cap 12 being removed. In FIG. 1B the device may be in a state ready to be operated, e.g. ready to perform a drug delivery operation when the operation is triggered. As depicted, the drug delivery device 100 further comprises a needle shroud 14. The needle shroud 14 protrudes distally from the housing 10 and/or was covered by the cap 12 when the cap was still attached to the housing 10. The needle shroud 14 is movable relative to the housing 10 from an initial position or first position to a second position or trigger position. The needle shroud 14 may be provided to extend beyond the tip of the needle which may protrude from the housing 10 before the drug delivery operation is commenced. The needle shroud 14 is movable in the proximal direction relative to the housing. During this movement, e.g. before the needle shroud reaches the second position, the needle may pierce the skin of the user. The needle shroud 14 can serve as a trigger member of the drug delivery device. The needle shroud as trigger member, when displaced proximally from the initial or first position depicted in FIG. 1B to the second or trigger position (see FIG. 1C), may automatically initialize the drug delivery operation, preferably when it is in the second position. The drug delivery operation can be initialized by removing a mechanical lock which prevents movement of the plunger rod in the distal direction or by moving plunger rod to disengage a mechanical lock via the moving needle shroud. Alternatively, the needle shroud when moved from the first position to the second position and expediently when in the second position may only enable triggering of the drug delivery operation. In this case, a separate trigger member, e.g. a trigger button on the proximal end of the housing 10, may be provided to initiate the drug delivery operation. Operating the trigger button to initiate the drug delivery operation may only be possible when the needle shroud 14 is in the second position. In yet another alternative, the needle shroud 14 may only be provided to prevent needle stick injuries before and/or after use of the drug delivery device 100. In this case, the needle shroud may be completely decoupled from the drive mechanism and/or not be involved in triggering or enabling triggering of the drug delivery operation at all.

The needle shroud 14 is provided to bear against the skin of the user during. Hence, the distal surface of the needle shroud 14 may provide a bearing surface 16. The bearing surface 16 may delimit and/or extend around a needle passage opening 17 provided in the needle shroud 14. The bearing surface 16 may be ring-like, circumferentially closed and/or be defined by an inward protrusion protruding radially from an inner wall of the needle shroud 14, e.g. a distal cylindrical portion thereof. The bearing surface 16 expediently is the distal end surface of the needle shroud 14.

FIG. 1C illustrates the needle shroud 14 in the second position relative to the housing 10. This is the position when the drug delivery operation has been initiated, can be initiated, and/or when the needle pierces the skin, for example. The needle 18 protrudes axially from the bearing surface 16 of the drug delivery device (particularly through the needle passage opening 17 in the shroud 14) and, by the distance with which it protrudes over the bearing surface, penetrates the skin (the skin is not shown in this representation). This distance may be characteristic for or be equal to the injection depth. The device is maintained in contact with the skin until the drug delivery operation has been completed, which may be indicated by an audible, tactile, and/or visual indication provided by the drug delivery device 100.

The drug delivery device can be designed to initiate the drug delivery operation at an injection depth which preferably is greater than or equal to one of the following values: 1 mm, 2 mm, 3 mm. Alternatively or additionally, the device can be designed to initiate the drug delivery operation at an injection depth which preferably is less than or equal to one of the following values: 7 mm, 6 mm, 5 mm. The injection depth at which the drug delivery operation is initiated (e.g. when the plunger is released) can be less than or equal to the maximum injection depth achievable with the device. The injection depth at which the drug delivery operation is initiated can be 5 mm+/−2 mm, where the variation may result from tolerances.

The maximum injection depth (e.g. when the needle shroud has reached its most proximal position relative to the housing) can be greater than or equal to: 6 mm, 7 mm, 8 mm. Alternatively or additionally, the maximum injection depth (e.g. when the needle shroud has reached its most proximal position relative to the housing) can be less than or equal to: 8 mm, 7 mm, 6 mm. The maximum injection depth can be 7 mm+/−1 mm, where the variation may result from tolerances.

Consequently, the range between the lowest depth for initiation (initiation depth) of the drug delivery operation and the highest maximum injection depth may be 3 mm to 8 mm.

After the drug delivery operation has been completed, the device may be removed from the skin (see FIG. 1D). The needle shroud 14 may be biased relative to the housing towards the first position by a spring (not shown). Thus, when the device is removed from the skin the needle shroud 14 is moved towards the first position with respect to the housing 10. The needle shroud 14 can be moved distally, e.g. beyond its first position, into a final, third or locked position relative to the housing 10. In this position the needle shroud 14 is expediently axially locked relative to the housing 10 against movement in the proximal direction, e.g. by a locking engagement between a locking feature of the shroud and the housing. As it is axially locked, the needle shroud can no longer be displaced proximally relative to the housing into the second position and/or into the first position. This protects the user from needle stick injuries after use. In this state, the device is locked out, see FIG. 1D.

The injection depth by which the needle of the drug delivery device pierces the skin is usually set by the design of the drug delivery device, which is designed to dispense drug from the medicament container having a specific needle, e.g. a needle of a specific length. However, there may be situations when the injection depth should be easily adjustable, while retaining the majority or all of the key parts of the drug delivery device. Particularly, the needle length and/or diameter may remain unchanged as may the general geometry of the medicament container. For example, autoinjectors for pediatric use may require a lower injection depth, simply because the tissue available for penetration with the needle is thinner for children than for adults. Therefore, herein, some concepts for drug delivery arrangements are proposed which allow modifications in the injection depth, e.g. reductions of the injection depth achieved by a drug delivery device of the arrangement, where the device may be one of the devices described further above.

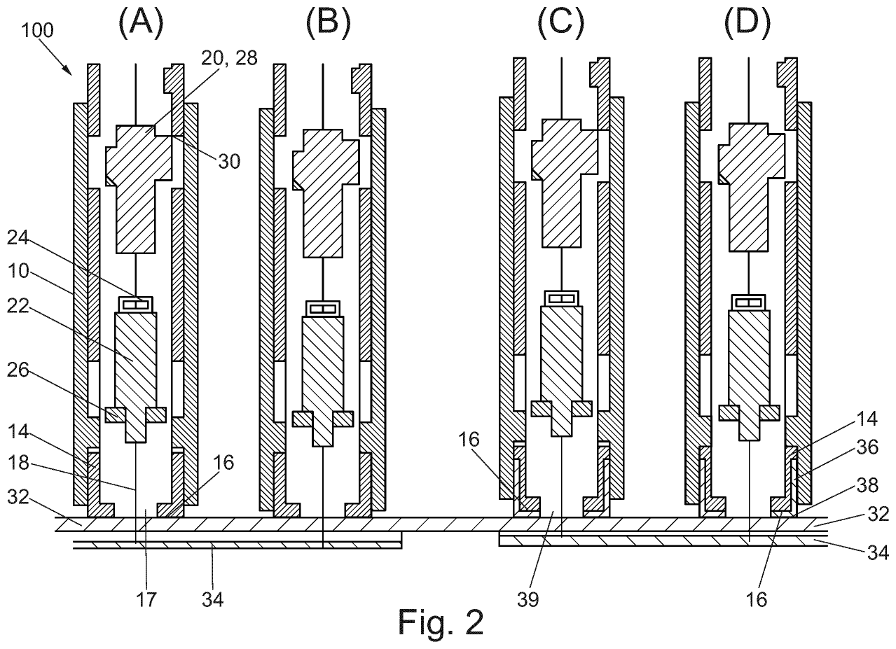

FIG. 2, in representations A through D, illustrates a concept for a drug delivery arrangement with a reduced injection depth. The concept is illustrated by way of an exemplary drug delivery device 100. The drug delivery device 100 is very similar to the drug delivery device described above and, especially, to the device disclosed in WO 2015/004052 A1, the entire disclosure content of which is incorporated herein by reference for all purposes, especially with respect to the design of the drive mechanism or “plunger release mechanism” as it is termed therein.

All of the representations A through D show a schematic sectional view through the device 100. The needle shroud 14 has been moved proximally relative to the housing 10 already to some extent in all of the representations. In the representations, the plunger rod 20 is shown as well as the medicament container 22, which, in this embodiment, is a syringe with the needle 18 at its distal end, e.g. a staked needle in a glass syringe body. The medicament container 22, at its proximal end, comprises a stopper 24 which sealingly closes the interior of the body of the medicament container 22 proximally. The stopper 24 is arranged to be driven in the distal direction relative to the body and/or towards the needle 18 in order to dispense drug from the interior of the medicament container, e.g. into the tissue of the user.

In the depicted embodiment, the medicament container 22 is supported axially within the housing 10. For this purpose, a medicament container support 26 is provided in the housing. The medicament container 22 may bear on the medicament container support 26, e.g. before commencement of the drug delivery operation, during the drug delivery operation and/or after completion of the drug delivery operation. The support 26 is secured to the housing against axial displacement in the distal direction (and preferably in the proximal direction) and/or is integrated into the housing 10, although this is not explicitly shown in the depicted schematic representation. Thus, in the presently depicted embodiment, the medicament container is fixed against displacement relative to the housing, preferably at least in the distal direction and more preferably also in the proximal direction. The medicament container 22 has a neck portion which provides a distally facing surface which can bear on a proximally facing surface of the support 26. The neck portion is closer to the distal end of the medicament container body of the medicament container from which the needle protrudes than to the proximal end of the medicament container body. The container 22 is distally supported in the housing 10. The device 100 also comprises a drive energy source 28, e.g. a drive spring, which is arranged to drive the plunger rod 20 in the distal direction once the drug delivery operation has been initiated.

Representations A and C show the situation of the drug delivery device 100 when the needle shroud 14 has been moved proximally, e.g. to initiate the drug delivery operation, but the plunger rod 20 is still locked, e.g. by engagement with the needle shroud 14. In the depicted embodiment the lock is highlighted by reference 30. The drive energy source 28 may be at least partially retained in the plunger rod 20 before the plunger rod 20 moves distally for the drug delivery operation. A drive spring as drive energy source 28 may bear against a distally facing surface of the housing which can be suitable to react the driving force exerted by the spring. The distally facing surface may delimit the housing proximally but is not shown in the schematic representations. Representations B and D illustrate the situation after distal movement of the plunger 20 is allowed, e.g. as it is no longer blocked by the mechanical lock 30, but right before it is commenced. In representations B and D, the needle shroud 14 is in the second or most proximal position relative to the housing 10. The second position may be defined by an abutment between the needle shroud 14 and a stop provided in the housing which limits proximal movement of the needle shroud 14 relative to the housing 10 (not explicitly shown). In all representation A to D the skin 32, e.g. the outermost layer of the skin, is shown as well as the tissue region 34 where the distal needle tip is placed for the drug delivery operation. In representations B and D, the needle has reached its end position in the tissue 34 while in representations A and C the needle still advances slightly through the skin as the needle shroud still moves proximally.

The drug delivery device 100 in representations A and B differs from the drug delivery arrangements depicted in figures C and D in that the drug delivery arrangement comprises an injection depth adjusting member 36 which is connected to the drug delivery device 100. The injection depth adjusting member 36 is connected to the needle shroud 14, expediently such that the injection depth adjusting member defines or provides the bearing surface 38 with which the drug delivery arrangement (or the modified device with the additional injection depth adjusting member) bears against the skin of the user. Hence, as opposed to representations A and B the injection depth is reduced in the arrangement of representations C and D by the thickness of the injection depth adjusting member 36 or the axial distance between bearing surfaces 38 and 16. The injection depth adjusting member 36 is connected to the distal end portion of the needle shroud 14, e.g. on the outer surface as depicted. The injection depth adjusting member 36 may be releasably or non-releasably connected to the needle shroud 14. For example, the injection depth adjusting member 36 can be clipped to the needle shroud, e.g. the distal end section thereof. The distal end section of the needle shroud 14 can have a cylindrical configuration. The injection depth adjusting member 36 comprises or defines a needle passage opening 39, which may be aligned with the needle passage opening 17 of the needle shroud 14. Both openings may have the same size and/or diameter. The drug delivery arrangement can be provided to the user with a yet not connected injection depth adjusting member 36 such that the user may decide whether he wants to use the drug delivery device with the regular injection depth, i.e. by using the bearing surface 16 as skin contact surface of the drug delivery device, or by attaching the injection depth adjusting member 36 which results in the bearing surface 38 as skin contact surface and in a reduced injection depth. Alternatively, the injection depth adjusting member 36 may already be connected to the drug delivery device, e.g. to the needle shroud 14, and the user can decide whether to remove it from the drug delivery device to increase the injection depth or whether the injection depth adjusting member 36 is kept in place and thereby defines smaller injection depth. The injection depth adjusting member may be permanently or releasably connected to the drug delivery device, e.g. to the needle shroud 14.

In the present disclosure, it is proposed to use an injection depth adjusting member 36 to adjust the injection depth of a drug delivery device 100, particularly to reduce the injection depth by a distance defined by the injection depth adjusting member. As will become apparent from the discussion of the further embodiments below, the injection depth may be fixed or may be varied, e.g. continuously or in discrete steps.

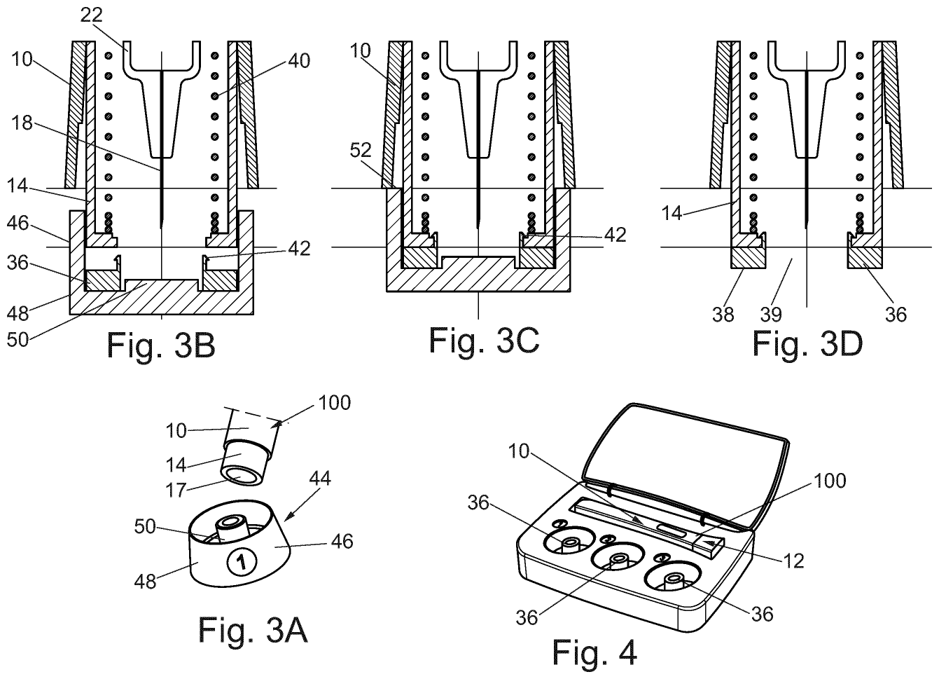

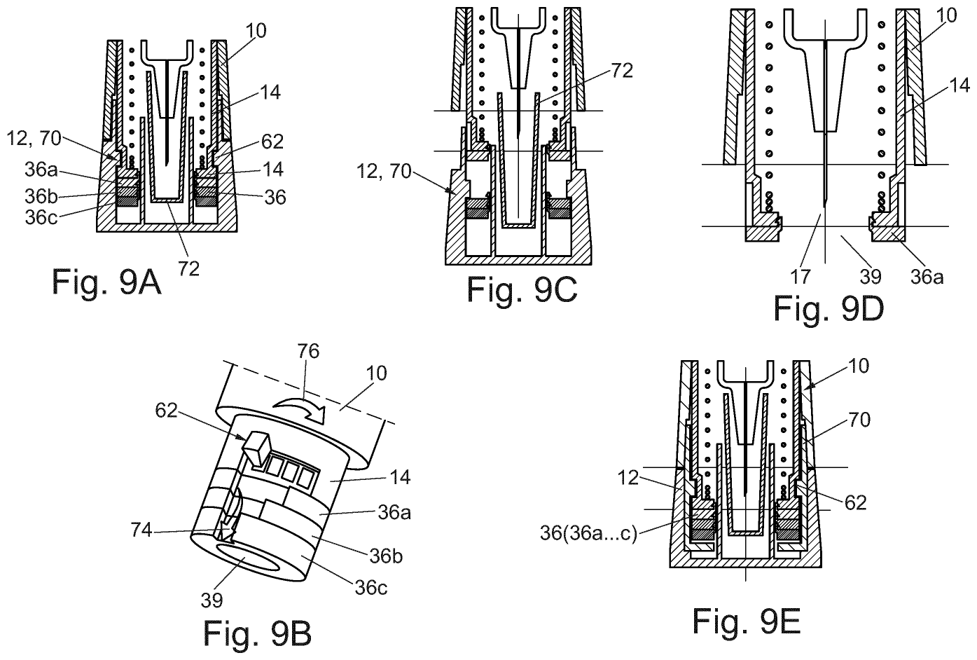

FIGS. 3A through 3D illustrate another embodiment of the drug delivery arrangement. The cap 12 and/or the needle shield (e.g. a rigid needle shield or RNS or a soft needle shield or SNS, see needle shield 72 in FIG. 9A, for example) may have already been removed from the needle 18 in the depicted situation. FIG. 3A illustrates the distal end portion of the drug delivery device 100 with the needle shroud 14 and the housing 10. Also depicted in FIG. 3 is the injection depth adjusting member 36 (see FIGS. 3B to D). The needle shroud 14 in its depicted first position extends distally beyond the needle tip in order to avoid unintentional needle stick injuries. In this embodiment, a shroud spring 40 is shown (which may be present in the other embodiments as well, of course). The spring 40 is expediently arranged to bias the needle shroud 14 distally relative to the housing 10. The spring 40 bears on an internal proximal surface of the needle shroud 14, e.g. a surface facing away from the bearing surface 16. Distal movement of the shroud 14 relative to the housing 10 beyond the first position may be prevented, e.g. by an interaction of the shroud with the plunger rod 20.

In the depicted embodiment, the injection depth adjusting member 36 has connection features 42, e.g. flexible clip arms or compliant beams. The needle shroud 14 has corresponding features, e.g. recesses which are adjusted to cooperate with the connection features 42 to establish a connection interface to connect the adjusting member 36 with the needle shroud 14. The connection features of the needle shroud 14 can be provided on or at the surface on which the shroud spring 40 bears, e.g. radially inwardly offset from the contact region between shroud spring 40 and the needle shroud 14. The connection may be releasable or non-releasable. If the connection is non-releasable, once the injection depth adjusting member 36 has been connected to the needle shroud 14, it stays connected thereto and cannot be removed, e.g. at least not without destroying one of the components which are connected to each other. The injection depth adjusting member 36 is a shim, from which the connection features axially protrude. The adjusting member 36 may be ring-like and the connection features 42 protrude axially from the ring in the proximal direction, preferably at an inner edge of the ring.

In the depicted embodiment, the injection depth adjusting member 36 is part of an injection depth adjusting assembly 44 with a plurality of components. The injection depth adjusting assembly 44 comprises, in addition to the injection depth adjusting member 36, a further member 46. The injection depth adjusting member 36 can be arranged and/or retained within the further member 46, preferably releasably retained, e.g. via a friction fit. The further member 46 may have an outer surface or exterior surface which delimits an interior of the assembly 44 wherein the adjusting member 36 is arranged in the interior, preferably in a manner such that it is not accessible from the exterior easily e.g. via the user's fingers. The further member 46 can comprise a protrusion 50, e.g. a central protrusion protruding from a distal end of the further member 46 and/or being arranged to be received in the opening of the injection depth adjusting member 36. The further member 46 is expediently configured to receive a portion of the needle shroud 14.

FIG. 3B illustrates the assembly 44 during the process of attaching the injection depth adjusting member 36 to the needle shroud 14. In FIG. 3B, the assembly has been guided over the needle shroud 14 such that the needle shroud 14 is partially received in the interior of the further member 46. The connection features 42 and the corresponding connection features of the needle shroud are still axially separated from each other. In FIG. 3C, injection depth adjusting member 36 has been connected to the needle shroud 14, e.g. by a snap-fit. The further member 46 may serve as an assembly jig for the connection of the injection depth adjusting member 36 to the shroud 14. The further member 46 expediently has a proximally facing surface 52 which is arranged to abut the housing 10, e.g. a distally facing surface thereof. The abutment between housing 10 and further member 46 is preferably established:
a) before the needle shroud 14 moves proximally, e.g. by the user during the connection process of the adjusting member 36 to the needle shroud 14; and/or
b) before the distal end of the needle shroud passes the needle tip in the proximal direction and/or after the needle shroud 14 moves proximally; and/or
c) before the needle shroud 14 reaches the second position and/or after the needle shroud passes the needle tip. Nevertheless, it is expediently ensured that the injection depth adjusting member 36 is connected to the needle shroud 14 before or at the latest when the further member 46 abuts the housing 10. In this way, it is prevented that the needle shroud 14 is accidentally moved proximally by the user via the further member 46 during the connection process. Hence, damages to the needle tip and/or accidental firing or triggering of the drive mechanism may be avoided during the connection process. Thus, by way of the abutment between the further member 46 and the housing 10, a reliable and safe connection process of the injection depth adjusting member 36 to the needle shroud 14 is facilitated. The abutment is depicted in FIG. 3C.

The force required to connect the injection depth adjusting member 36 to the needle shroud 14 is expediently smaller than the force required to move the needle shroud proximally into the second position relative to the housing, e.g. against the force of spring 40. A slight movement of the needle shroud proximally away from the first position, e.g. against the force of spring 40, may occur during connection of the injection depth adjusting member although this is not expressly shown. The force required to connect the injection depth adjusting member 36 to the needle shroud 14 may be lower than the force required to displace the needle shroud 14 relative to the needle 18 such that the needle tip protrudes distally from the needle shroud 14. This can be assisted by designing the connection features 42 and the corresponding connection features appropriately. Once the injection depth adjusting member 36 has been connected to the needle shroud 14, which, to the user, may be indicated by the abutment between the further member 46 and the housing 10, the further member 46 can be removed leaving the injection depth adjusting member in position and connected to the needle shroud 14. Hence, during the subsequent drug delivery operation, the injection depth can be defined by the injection depth adjusting member 36 and the bearing surface 38 of this member may bear against the skin, see FIG. 3D. The further member 46 may facilitate the assembling of the adjusting member 36 to the needle shroud 14 but can also be dispensed with. That is to say, the injection depth adjusting member 36 could also be directly touched by the user in order to assemble it to the needle shroud 14.

The injection depth adjusting member 36 in the presently proposed concepts is adjusted to the needle shroud 14 such that the needle shroud is movable relative to the housing 10 in the desired manner or in the manner required for operation of the drug delivery device. For example, the outer diameter of the injection depth adjusting member may be adjusted to the outer diameter of the needle shroud. The inner diameter of the injection depth adjusting member may be adjusted to the inner diameter of the needle shroud and/or to the needle such that the needle may still pass through the opening in the adjusting member to pierce the skin. The adjusting member 36 may be arranged outside of the housing 10 in every possible position of the needle shroud 14 relative to the housing 10.

FIG. 4 illustrates an embodiment of a drug delivery arrangement. The arrangement comprises the drug delivery device 100 with the housing 10 and the cap 12. Moreover, the arrangement comprises a plurality of injection depth adjusting members 36. Each member may be configured to achieve a different injection depth (or depth reduction). The device 100 may be usable without any injection depth adjusting member connected to the device as well, which may result in the greatest injection depth. Each of the adjusting members 36 may be attachable to the needle shroud 14 of the device (not shown in this embodiment). In case the injection depth adjusting member 36 is provided in the form of an assembly 44, each assembly may be configured as has been previously described in conjunction with FIGS. 3A to 3D. The device 100 and injection depth adjusting members 36 may be arranged in a common case 51. Different adjusting members 36 may be differently coded, e.g. color-coded, to signal to the user which one of the adjusting members 36 achieves the desired injection depth. The case may comprise information on which code corresponds to which desired injection depth.

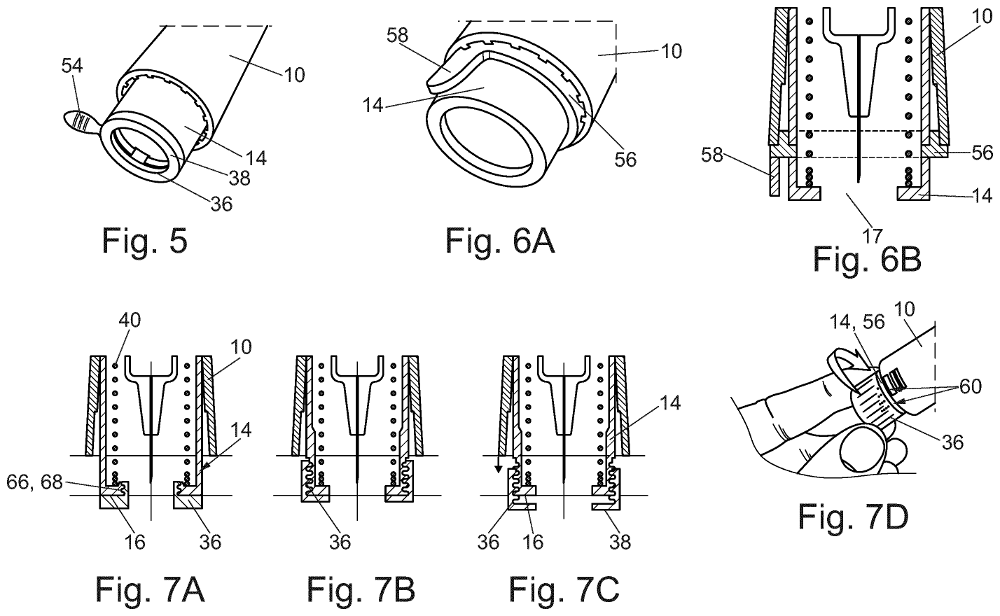

FIG. 5 illustrates another embodiment of a drug delivery arrangement. In FIG. 5, the injection depth adjusting member 36 is pre-installed or connected to the needle shroud 14. The injection depth adjusting member 36 is preferably releasably connected to the needle shroud 14. The member 36 may be removed by the user in order to achieve a greater injection depth, e.g. thereby exposing the bearing surface 16 of the needle shroud 14 (not explicitly shown in FIG. 5). In order to facilitate removal, the injection depth adjusting member 36 may comprise a tab 54. The tab 54 may be gripped by the user in order to remove the injection depth adjusting member 36 from the needle shroud 14. When the cap 12 is in place, the tab 54 may be deflected, e.g. to be oriented in the axial direction. The tab 54 may assume the radially protruding shape depicted in FIG. 5 only when the cap 12 is removed to expose the needle shroud 14 and/or the injection depth adjusting member 36, e.g. on account of the elasticity of the tab 54.

FIGS. 6A and 6B illustrate another embodiment on the basis of a perspective view in FIG. 6A and a schematic sectional view in FIG. 6B. Here, a safety member 56 is connected to the needle shroud 14 (after the cap 12 has been removed already). The safety member 56, expediently, is removable from the needle shroud 14. The safety member 56 is expediently removed after the injection depth adjusting member 36 (not shown in FIGS. 6A and 6B but may nevertheless be present) has been connected to the needle shroud 14. The safety member 56 is arranged to prevent proximal movement of the needle shroud 14, e.g. by cooperating with the needle shroud 14 and the housing 10, unless it is removed. To facilitate removal, a tab 58 may be provided which can be gripped by the user in order to remove the safety member 56. The safety member 56 may protrude radially from an outer surface of the needle shroud 14 when connected to the needle shroud 14. The safety member 56 may be ring-like. The needle shroud 14 can protrude through the ring. A proximally facing surface of safety member 56 abuts or is arranged to abut a distally facing surface of the housing 10 and/or a distally facing surface of the safety member 56 is arranged to abut or abuts a proximally facing surface of the needle shroud 14. The safety member 56 preferably prevents or restricts movement of the needle shroud 14 in the same manner as describe above for the abutment between the further member 46 and the housing (see a) to c) further above). Thus, needle stick injuries and/or accidental firing of the drive mechanism may be prevented. The safety member 56 may be implemented in any one of embodiments disclosed herein. It is particularly expedient to include the safety member, in case the injection depth adjusting member is available for manipulation, such as for removal or movement relative to the needle shield. By providing the additional safety member 56, it can be avoided that the device fires accidentally or the user is pierced with the needle while the user still manipulates the injection depth adjusting member 36. It is also conceivable that the safety member 56 is configured as a injection depth adjusting member which has to be displaced from a first position or non-operating position relative to the needle shroud into a operating position e.g. to provide a distal bearing surface 38. Such an embodiment is not expressly shown.

FIGS. 7A to 7D illustrate further embodiments of the drug delivery arrangement. The drug delivery arrangement, in each case, comprises an injection depth adjusting member 36. The adjusting member 36 may be preinstalled at the drug delivery device 100 (connected to the device member) or installable by the user. The injection depth adjusting member 36, in each case, is connected to a distal end portion of the needle shroud 14. The injection depth adjusting member 36 further is movably connected or connectable to the needle shroud 14. The respective injection depth adjusting member 36 is threadedly engaged or engageable with the needle shroud 14. In FIG. 7A, the needle shroud 14 has a thread 66 on an inner diameter, e.g. on an inner diameter of the needle passage opening, such as in the region which may adjoin the bearing surface 16 provided by the needle shroud 14. This thread may be threadedly engaged by the injection depth adjusting member, e.g. by a mating thread 68, such as an outer thread. In FIG. 7B, the threaded engagement is established by way of an outer thread 66 of the needle shroud 14 and, preferably, a mating inner thread 68 of the injection depth adjusting member 36.

By screwing the injection depth adjusting member 36 relative to the needle shroud 14 the injection depth may be adjusted, e.g. decreased or increased. Specifically, the bearing surface 38 provided by the injection depth adjusting member 36 can be displaced axially away from the bearing surface 16 provided by the bearing and/or distally relative to the needle shroud 14 when rotating the member 36 in one direction relative to the needle shroud 14. In this way, the injection depth can be further reduced. By moving the injection depth adjusting member in the opposite axial direction, e.g. by rotation in the opposite direction, the depth may be increased again, e.g. until the most proximal position of the adjusting member is reached. If the injection depth adjusting member is disconnected from the needle shroud 14, e.g. by unscrewing it from the needle shroud 14, the needle shroud 14 can be used with its bearing surface 16 for contacting the skin for a drug delivery operation. The threaded connection between the needle shroud 14 and the injection depth adjusting member 36 is, preferably, a self-locking threaded connection. In this way, the activation force or displacement force required to displace the needle shroud 14 from the first position in the proximal direction into the second position, which force may comprise contributions of the spring force of the spring 40, does not result in relative rotational movement between the injection depth adjusting member and the needle shroud 14 although it may act on the threaded connection. In FIG. 7C, the injection depth adjusting member has been moved into the distal direction to further reduce the injection depth.

In the embodiment depicted in FIGS. 7A to 7D the injection depth may be varied continuously or may be selected from a fixed number of injection depths. A fixed number of injection depths may be indicated by indications on the device or the arrangement, e.g. by alignment of indications on the needle shroud 14 (or the safety member 56) and the injection depth adjusting member 36. This is illustrated in FIG. 7D by indications 60. The injection depth adjusting member 36 may be manually manipulated by the user in the depicted embodiment.

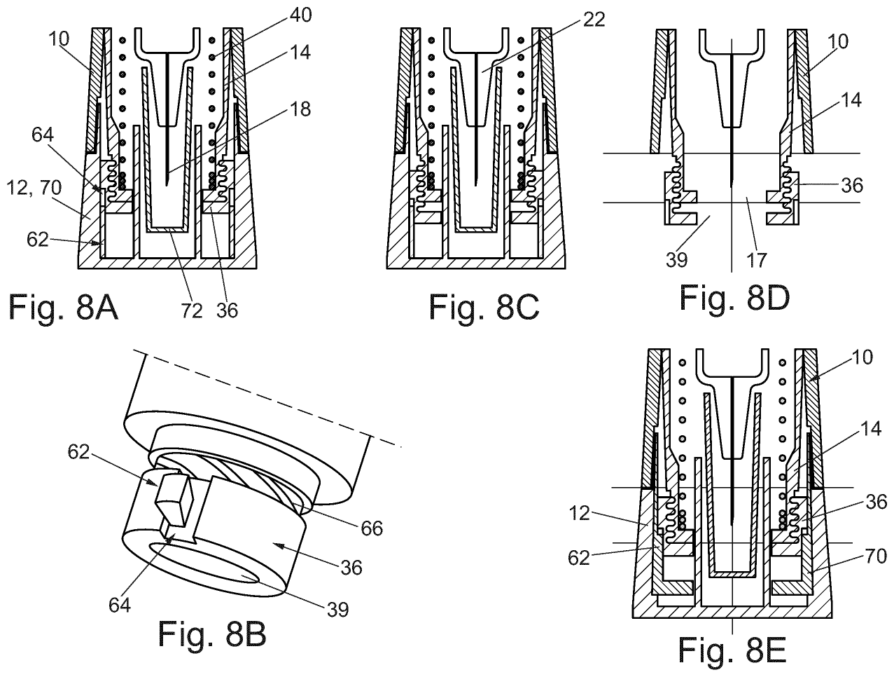

FIGS. 8A through 8E illustrate further embodiments of a drug delivery arrangement. This embodiment is very similar to the ones discussed in conjunction with FIGS. 7A to 7D. Hence, the following focuses on the differences. In this embodiment, the injection depth is not adjusted manually by manipulating directly the injection depth adjusting member 36. Rather, the injection depth adjusting member is operatively coupled to a selection member 70. The selection member 70 is provided to be manipulated by the user in order to select the desired injection depth. The selection member 70 is the cap 12 in the embodiment shown in FIGS. 8A to 8D. However, a separate selection member 70 in addition to the cap 12 is also possible (see further below, e.g. in FIG. 8E). FIGS. 8A through 8C show different stages during the adjustment or selection process for adjusting the injection depth to a desired injection depth. The selection member 70 is rotationally locked to the injection depth adjusting member 36. For this purpose, the selection member 70 or cap 12 has one or more interface features or selection features 62, e.g. spline features, such as one or more ridges or one or more bosses. Feature(s) 62 is (are) provided to operatively couple the selection member 70 to the injection depth adjusting member 36, e.g. to engage a corresponding interface feature 64 of the adjusting member 36, e.g. a longitudinally or axially (i.e. distally and/or proximally) oriented slot. FIG. 8B shows just the features 62 and 64 without the remainder of the selection member 70 or cap 12.

As the selection member or cap 12 is coupled, e.g. rotationally locked, to the injection depth adjusting member 36, rotation of the selection member 70 is transferred to the injection depth adjusting member 36 and moves, e.g. rotates, the injection depth adjusting member 36 relative to the needle shroud 14 until the desired injection depth is reached. The selection member 70 or cap 12 is arranged relative to the needle shroud 14 and/or the injection depth adjusting member 36 such that, when selection member 70 or cap 12 is rotated relative to the housing 10, it does not change its relative position relative to the needle shroud 14 and/or the housing 10. In other words, the selection member 70 or cap 12 is axially locked to and rotatable relative to the housing 10. The injection depth adjusting member 36, however, upon rotation of the selection member 70 or cap 12 moves relative to the needle shroud 14 in an amount corresponding to or determined by the pitch of the thread which couples the adjusting member 36 with the needle shroud 14 threadedly, e.g. thread 66 or 68. For example, the selection member 70 or cap 12 may have one or more compliant arms which engage a circumferentially disposed ring-like recess in the housing. Indications on the outside of the housing and/or the cap 12 may indicate the current injection depth (not shown). Once the desired injection depth has been selected (see FIG. 8C), the selection member 70 or cap 12 may be removed, e.g. by pulling the cap in the distal direction relative to the housing 10 and/or relative to the injection depth adjusting member 36 (see FIG. 8D). The movement to remove the selection member 70 or cap 12 is permitted by the splined coupling between the injection depth adjusting member 36 and the selection member 70 or cap 12.

In the embodiments depicted in FIGS. 8A through 8E, the needle shield 72, which covers the needle 18 when the cap 12 is attached to the drug delivery device 100 is shown. The needle shield 72 may be axially and/or rotationally locked to the cap 12 such that it is removed together with the cap 12, e.g. by prongs or barbs provided in the cap (not shown). When the cap 12 is rotated, the needle shield 72 may rotate as well relative to the housing 10 and/or the needle shroud 14. The medicament container 22 may rotate as well relative to the housing 10 or be rotationally locked relative to the housing e.g. by according rotational stops. If the medicament container rotates together with the needle shield 72, there is no relative rotational movement between the needle shield 72 and the needle 18, such that the risk of damages to the needle 18 may be reduced.

The cap 12 may be directly engaged with the injection depth adjusting member 36 as depicted in FIGS. 8A through 8D. Alternatively, the cap 12 can be indirectly coupled to the injection depth adjusting member 36 or be decoupled from the injection depth adjusting member 36. This option is depicted in FIG. 8E. For this purpose, a selection member 70 may be provided in addition to the cap 12. The member 70 is arranged at least partially in the interior of the cap 12. The selection member 70 is inaccessible from the exterior of the device when the cap 12 is in place. The cap 12 can be removed from the device, thereby leaving the selection member 70 in place. The cap 12 can be decoupled from the selection member such that rotation of the cap is not transferred to the selection member or the cap 12 may be rotationally locked relative to the housing 10 and/or be removable from the housing by pulling (e.g. without rotation). Hence, the cap 12 can be removed from the device 100 in the usual manner, together with the needle shield 72, e.g. without involving rotation of the needle shield relative to the housing 10 and/or the needle shroud 14. The selection member 70 is expediently rotationally locked to the injection depth adjusting member 36 and/or and can be manipulated, e.g. rotated, until the desired injection depth is reached after removal of the cap 12. When the desired injection depth has been set, the selection member 70 can be removed from the device as well. The selection member 70 is expediently arranged to block or prevent movement of the needle shroud 14 relative to the housing 10 and/or to prevent access to the needle shroud 14 (e.g. via the injection depth adjusting member 36) before the member 70 is removed from the device 100. This increases the safety for the user or reduces the risk of accidental firing or triggering during injection depth adjustment. After the selection member 70 depicted in FIG. 8E has been removed, the device has the configuration depicted in FIG. 8D.

Having the selection member 70 and/or the cap 12 in place until the desired injection depth is reached may have the advantage that the user is protected from needle stick injuries throughout the injection depth adjusting process as compared to cases where the user direction manipulates the injection depth adjusting member.

FIGS. 9A through 9E illustrate further embodiments of the drug delivery arrangement. As in the previously discussed embodiments, a selection member 70 is provided which is movable relative to the housing 10 and/or the needle shroud 14 in order to select a desired injection depth. Hence, features which have been described previously do also apply for this embodiment. As in the previously described embodiment, the cap 12 may act as selection member 70 or a separate selection member 70 may be provided which is decoupled from the cap 12. In the latter case, the cap 12 can be removed (together with the needle shield 72) and the selection member 70 is made available for user manipulation. Again the needle shield 72 may be axially and/or rotationally locked to the cap 12. The selection member 70 may be or may comprise a sleeve (as in the previous embodiment).

In the depicted embodiment, an injection depth adjusting member 36 is connected to the needle shroud 14. As depicted, a plurality of injection depth adjusting members 36a to 36c are arranged in a stacked manner. However, it will be appreciated that the presently discussed concept also works with just one injection depth adjusting member 36 which is connected or preinstalled to the needle shroud 14. If several injection depth adjusting members are provided as depicted, these members are preferably interconnected. Particularly, member 36c is preferably connected to the next member 36b. Member 36b may be connected to member 36a. Member 36a may be connected to the needle shroud 14. Associated connection features, e.g. snap features may be provided for the respective connection, which, for example, may be releasable (but preferably not re-establishable). The selection member 70 comprises a selection feature 62, e.g. a radially inwardly protruding boss. The members 36a to 36c may form an injection depth adjusting member assembly, which is connected to or preinstalled to the needle shroud 14. The injection depth adjusting members of the assembly may be selectively detachable from the assembly or from the needle shroud. The selection member 70 is configured to selectively detach one or more members of the assembly simultaneously with its removal depending on its rotational orientation relative to the injection depth adjusting member(s). One or more members of the assembly may remain after removal of the selection member.

FIG. 9B illustrates the selection feature 62 without the remainder of the selection member 70. The selection feature 62 is arranged such that depending on the rotational position of the selection feature 62 relative to the needle shroud 14 or the injection depth adjusting member (assembly), the selection feature, when moved in the distal direction, which is indicated by the arrow 74, detaches one or more injection depth adjusting members 36 from the needle shroud 14 (or the assembly) or does not detach any injection depth adjusting member from the needle shroud (or the assembly). If, in the position depicted in FIG. 8B, selection member 70 or cap 12 is moved distally, e.g. to detach cap 12 or selection member 70 from the device, none of the injection depth adjusting members is removed and the injection depth is the lowest depth achievable with this arrangement. This is because a slot extends all the way through all of the injection depth adjusting members and the selection feature travels distally within the slot and does not interact with any one of the injection depth adjusting members in a way to disconnect that member from the needle shroud 14. The injection depth can be increased stepwise by rotating the selection feature 62 or the selection member 70 relative to the needle shroud 14 and/or the injection depth adjusting member(s) 36 (e.g. clockwise from the position depicted in FIG. 9B). In FIG. 9B, three further discrete relative angular or rotational positions relative to the needle shroud 14 or the injection depth adjusting members are possible, e.g. by rotating the selection member 70 accordingly. In each relative position, a different desired injection depth is achieved as one, two, or three (all) injection depth adjusting members 36a to 36c are detached or removed from the needle shroud 14 when the selection member 70 is removed depending on the relative rotational position. This is because the selection feature travels in a recess or slot along the adjusting members which remain connected to the needle shroud 14 and engages a proximally facing surface of the next injection depth adjusting member. Then, this injection depth adjusting member is disconnected from the needle shroud 14 together with all of the injection depth adjusting members arranged distally from the injection depth adjusting member which the selection feature 62 engages.

In the situation depicted in FIG. 9C, the selection member 70 or cap 12 has been rotated to the third position as seen from the first position depicted in FIG. 9B. When, subsequently, the selection member 70 or cap 12 is detached, injection depth adjusting members 36b and 36c are detached as well. Thus, only one injection depth adjusting member remains, i.e. injection depth adjusting member 36a. Of course, it should be appreciated that the operation principle also works with only one injection depth adjusting member which is either removed from the needle shroud 14 or not depending on the rotational position of the selection member relative to the adjusting member and/or the needle shroud 14. Also, the principle works with two or more than three injection depth adjusting members where, dependent on the relative rotational position of the selection feature 62 to the needle shroud 14 or the injection depth adjusting member(s) selected injection depth adjusting member(s) remain connected to or are removed from the needle shroud 14. The rotatability of the selection feature 62 relative to the needle shroud 14 and/or the injection depth adjusting member(s) is indicated by the arrow 76 in FIG. 9B.

FIG. 9D shows the situation after the selection member 70 or cap 12 has been detached or removed from the device and only the injection depth adjusting member(s) for the desired injection depth remain (here the member 36a) or just the needle shroud 14 remains (not shown).

FIG. 9E illustrates an embodiment which uses a selection member 70 with a selection feature 62 which is decoupled from the cap 12 such that (as in the embodiments of FIG. 8E) the cap 12 may be removed (together with the needle shield 72) independently from the selection member.

In the present disclosure, the thickness of one injection depth adjusting member or the reduction in injection depth provided by one injection depth adjusting member can be greater than or equal to one of the following values: 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm. Alternatively or additionally, the thickness of one injection depth adjusting member or the reduction in injection depth provided by one injection depth adjusting member can be less than or equal to one of the following values: 3.0 mm, 2.5 mm, 2.0 mm, 1.9 mm, 1.8 mm, 1.7 mm, 1.6 mm, 1.5 mm. These dimensions might be particular suitable for arrangements having just one injection depth adjusting member. Alternatively they may relate to the respective injection depth adjusting member of an injection depth adjusting assembly or to the thickness of the entire assembly. If plastic components are used for the adjusting members, the reduction is preferably greater than or equal to 0.5 mm. Smaller reductions can be reached using metal components for the adjusting members.

In case an injection depth adjusting member assembly is provided, the respective member may have a thickness of greater than or equal to or the reduction in injection depth provided by the respective injection depth adjusting member may be greater than or equal to one of the following values: 0.1 mm, 0.2 mm, 0.3 mm, 0.4 mm, 0.5 mm, 0.6 mm, 0.7 mm, 0.8 mm, 0.9 mm, 1 mm. Alternatively or additionally, the thickness of the respective member may be less than or equal to or the reduction in injection depth provided by the respective injection depth adjusting member may be less than or equal to one of the following values: 1.5 mm, 1.4 mm, 1.3 mm, 1.2 mm, 1 mm.

As an example, one could use increments of 0.5 mm for the three adjusting members of the assembly in FIG. 9A et seq, resulting in a range of injection depths (lowest initiation depth to highest maximum depth) of 2-7 mm (all adjusting members in place), 2.5-7.5 (one removed), 3-8 mm (two removed), and 3.5-8.5 mm (all removed, needle shroud face is used to bear on the skin).

As another example which targets a wider range, and could employ plastic parts, one could use increments of 1 mm for the 3 adjusting members targeting depths of 2-7 mm (all members in place), 3-8 (one removed), 4-9 mm (two removed), and 5-10 mm (all removed, and needle shroud face used). This example would allow for deeper injections in those with high subcutaneous fat layers in certain areas.

In case the reduction in injection depth is continuously adjustable (as in the FIG. 8 embodiment), the depth range may be 2 mm to 7 mm (lowest initiation depth to highest injection depth).

For devices where the adjusting member might be selected, e.g. FIGS. 3A to D and 4, the adjusting members could also use increments of 0.5-1 mm with the depth range possible being 2-7 mm.

For devices with one preinstalled adjusting member, e.g. as in FIGS. 5, 6A and B, the installed adjusting member could target the injection depth range of 2-7 mm, and with the member not in place, 2.5 mm-7.5 mm or deeper could be implemented.

Maximum injection depths of less than or equal to the following values may be suitable for children and/or grown-ups: 11 mm, 10 mm, 9 mm, 8 mm, 7 mm, 6.5 mm, 6 mm, 5.5. mm, 5 mm. The ideal injection depth might depend on subcutaneous fat layers (if fat layers are significant, higher injection depths are suitable) Alternatively or additionally, the injection depth for children or grown-ups should be greater than or equal to one of the following values: 2 mm, 2.5 mm, 3 mm, 3.5 mm, 4 mm, 4.5 mm, 5 mm. The presently proposed arrangement may achieve such injection depths by one or more injection depth adjusting member(s).

The maximum injection depth of a non-modified drug delivery device, i.e. without an injection depth adjusting member being connected to the device, may be greater than the values mentioned for the lower limit for the injection depth suitable for children specified above and/or may be 11 mm, 10 mm, 9 mm, 8 mm or 7 mm, for example.

The drug delivery arrangement may be configured such that, when the injection depth adjusting member has been released or disconnected from the drug delivery device, it cannot be reconnected, e.g. by providing a connection feature which breaks when the injection depth adjusting member is removed from the drug delivery device. This has the advantage that the user knows that he cannot attempt to reconnect the adjusting member. When the adjusting member has been removed, the device may be ready to initiate a drug delivery operation and one or more safety mechanisms may no longer be present. Hence, preventing the user from an attempt to reattach the adjusting member, which might be dangerous, may increase user safety.

There are also devices, in which the housing itself bears against the skin during the drug delivery operation, i.e. the device member providing the member bearing surface is the housing. In this case, the needle shroud may be only moved to cover the needle after the drug delivery operation has been completed or be dispensed with entirely. Hence, references above to the needle shroud should be understood to also refer to a general device member (potentially different from the needle shroud 14) which is configured to bear against the skin with a member bearing surface during the drug delivery operation. The proposed injection depth adjusting member can be connected or connectable to the device member as described further above. The injection depth may, particularly, also be adjusted for devices with movable containers or needles by the disclosed concepts.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Any disclosure described herein is not limited by the description in conjunction with the exemplary embodiments. Rather, the disclosure comprises any new feature as well as any combination of features, particularly including any combination of features in the patent claims, even if said feature or said combination per se is not explicitly stated in the patent claims or exemplary embodiments.

1-22. (canceled)

23. A drug delivery arrangement, comprising:
a drug delivery device comprising a device member and a housing adapted to receive a needle, the needle being configured to pierce a skin;
an injection depth adjusting member that is connectable to or connected to the device member of the drug delivery device;
wherein the injection depth adjusting member is configured to provide an adjuster bearing surface with which the drug delivery arrangement bears on the skin during use when the injection depth adjusting member is connected to the device member; and
wherein when the injection depth adjusting member is connected to the device member, the injection depth adjusting member is configured to define an injection depth by which the needle can penetrate the skin.

24. The drug delivery arrangement of claim 23, wherein when the drug delivery device is operated to perform a drug delivery operation, the device member is arranged to provide a member bearing surface with which the drug delivery device can bear on the skin.

25. The drug delivery arrangement of claim 23, wherein the injection depth adjusting member is releasably connected to the device member.

26. The drug delivery arrangement of claim 25, wherein the drug delivery arrangement comprises a plurality of injection depth adjusting members that are releasably connected to the device member.

27. The drug delivery arrangement of claim 23, wherein the injection depth adjusting member is configured to be disconnected from the drug delivery device and connected to the device member.

28. The drug delivery arrangement of claim 27, comprising a plurality of injection depth adjusting members that are configured to provide different injection depths when connected to the device member.

29. The drug delivery arrangement of claim 28, wherein the plurality of injection depth adjusting members form an injection depth adjusting member assembly that is connectable to or connected to the device member of the drug delivery device.

30. The drug delivery arrangement of claim 29, wherein when the injection depth adjusting member assembly is connected to the device member, the plurality of injection depth adjusting members is configured to define the injection depth by which the needle can penetrate the skin.

31. The drug delivery arrangement of claim 29, wherein each injection depth adjusting member of the injection depth adjusting member assembly is configured to provide an adjuster bearing surface with which the drug delivery arrangement bears on the skin during use when the respective injection depth adjusting member is exposed.

32. The drug delivery arrangement of claim 29, comprising a selection member configured to selectively detach one or more of the injection depth adjusting members simultaneously with its removal depending on its rotational orientation relative to the injection depth adjusting members, such that the one or more of the injection depth adjusting members of the injection depth adjusting member assembly remain after removal of the selection member.

33. The drug delivery arrangement of claim 23, wherein the device member is a member different from the housing and movable relative to the housing.

34. The drug delivery arrangement of claim 33, wherein the drug delivery device comprises a removable safety member that is operatively connected to the device member to prevent movement of the device member relative to the housing when the safety member is connected to the device member.

35. The drug delivery arrangement of claim 23, wherein at least one of the device member is a needle shroud or the needle is axially fixed relative to the housing.

36. The drug delivery arrangement of claim 23, wherein the drug delivery device comprises a medicament container holding a medicament.

37. The drug delivery arrangement of claim 23, wherein when the injection depth adjusting member is connected to the device member, the injection depth adjusting member is rigidly connected to the device member.

38. The drug delivery arrangement of claim 37, wherein the injection depth adjusting member is threadedly connected to the device member and rotatable relative to the device member; and
wherein a threaded connection between the device member and the injection depth adjusting member is self-locking.

39. The drug delivery arrangement of claim 23, comprising a selection mechanism, which is operable to select a desired injection depth from a plurality of possible injection depths;
wherein the selection mechanism comprises a selection member which is rotatable from a first position to a second position; and
wherein the first position is a position for a first injection depth and the second position is a position for a second injection depth.

40. The drug delivery arrangement of claim 39, wherein the selection member is removable from the drug delivery device;
wherein, in the first position, the selection member is arranged such that the injection depth adjusting member is removed from the device member together with the selection member; and
wherein, in the second position, the selection member is decoupled from the injection depth adjusting member such that the selection member is removed from the device member and the injection depth adjusting member stays connected to the device member.

41. The drug delivery arrangement of claim 32, wherein the drug delivery device comprises a drive energy source, and is configured to perform a drug delivery operation using the energy obtainable from the drive energy source; and
wherein the drug delivery device is an autoinjector.
